(19) United States
(12) Patent Application Publication
(10) Pub. No.: US 2017/0022200 A1
(43) Pub. Date: Jan. 26, 2017

(54) POLYMORPHIC FORMS OF 4,5-DIHYDRO-1H-PYRROLO[2,3-F]QUINOLINE-2,7,9-TRICARBOXYLIC ACID AND ITS DISODIUM SALT, PROCESS FOR THEIR PREPARATION AND THEIR USE

(71) Applicant: ANTHEM BIOSCIENCES PRIVATE LIMITED, Bangalore, Karnataka (IN)

(72) Inventors: RAJULU GAVARA GOVINDA, Bangalore (IN); GANESH SAMBASIVAM, Bangalore (IN); TOM THOMAS PUTHIAPARAMPIL, Bangalore (IN); RAVINDRA CHANDRAPPA KORAMANGALA, Bangalore (IN)

(73) Assignee: ANTHEM BIOSCIENCES PRIVATE LIMITED, Bangalore, Karnataka (IN)

(21) Appl. No.: 15/302,720

(22) PCT Filed: Apr. 15, 2015
(86) PCT No.: PCT/IB2015/052748

§ 371 Date: Oct. 7, 2016

(30) Foreign Application Priority Data
Apr. 16, 2014 (IN) 1988/CHE/2014

(51) Int. Cl. C07D 471/04

(52) U.S. Cl. CPC C07D 471/04; C07B 2200/13

(57) ABSTRACT

The present disclosure relates to polymorphic form of PQQ and/or its salts represented by formula (I), wherein “n” and “m” are selected from a group consisting of: (a) “n”=3, “m”=0 and (b) “n”=1, “m”=2; and; R3 is Na+. The present disclosure also relates to a process for preparing the polymorphic form of compound of formula (I) and/or its salts, a composition comprising the polymorphic form of compound of formula (I) and/or its salts and use thereof.

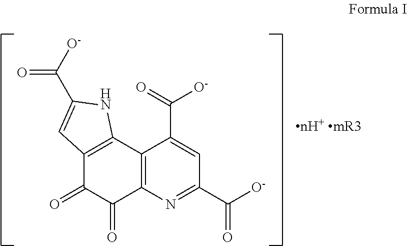

### TECHNICAL FIELD

The present disclosure is in the field of pharmaceutical and chemical sciences. The present disclosure relates to polymorphic form of pyrroloquinoline quinone (PQQ) and/or its salts represented by formula I

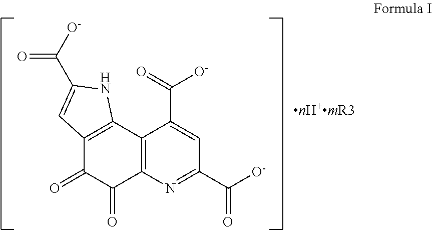

wherein “n” and “m” are selected from a group consisting of: (a) “n”=3, “m”=0 and (b) “n”=1, “m”=2; and

### R3 is Na⁺.

The present disclosure also relates to a process for preparing polymorphic form of compounds of formula I, a composition comprising polymorphic form of compounds of formula I and use thereof.

### BACKGROUND AND PRIOR ART

Pyrroloquinoline quinone (PQQ) is a natural product and is categorized as an essential micronutrient and dietary supplement as it plays a critical role in the mitochondrial biogenesis. PQQ is also known as methoxatin. Among many applications, primary uses of PQQ are to protect mitochondria from oxidative stress, providing neuroprotection and cardioprotection. Common food sources of PQQ are parsley, green pepper, green tea, papaya, kiwi, milk and tofu. However, the available concentrations of PQQ in the food sources are only in picomolar (pM) to nanomolar (nM) levels. This necessitates the development of chemical processes which can produce large quantities of PQQ and its salts.

Few reports are known (J. Am. Chem. Soc. 103 (1981), 5599-5600; Helv, Chem, Acta 76 (1993), 1667; WO2006/102642A1, JP 7-113024 A) in literature to produce PQQ by chemical method as a free acid and its salts. However, the synthesis involves large number (9-10) of steps and the isolation of advanced intermediates and final product further involves tedious workup procedure.

Several research groups have reported different polymorphs of PQQ with or without metal salts and process for preparation of PQQ polymorphs. Polymorphism is the ability of a chemical/pharmaceutical compound in the solid state to exist in different crystalline forms having the same chemical composition with modified physical properties and varied biological applications. Identification of new polymorphic forms of therapeutically important chemical molecules is an important step in the drug development process.

Recently, Junichi EDAHIRO et al (US 20120116087 A1) reported the defined crystal structure of PQQ di and tri sodium salts. However, the final isolation involved usage of organic solvents. Furthermore, alcoholic solvents are known to form adducts with PQQ.

Hence, it can be observed that there is an immense need for better and simpler synthetic routes for obtaining Pyrroloquinoline quinone (PQQ) salts. The present disclosure aims at overcoming the drawbacks of prior art and provide with stable crystalline forms of PQQ and its salts by improved, cost effective and scalable synthetic routes having minimal steps from advance intermediate.

### BRIEF DESCRIPTION OF ACCOMPANYING DRAWINGS

The features of the present disclosure will become more fully apparent from the following description taken in conjunction with the accompanying drawings. Understanding the drawings depict only several embodiments in accordance with the disclosure and are therefore, not to be considered limiting of its scope, the disclosure will be described with additional specificity and detail through use of the accompanying drawings:

FIG. 1 illustrates the powder XRD spectra of polymorph-1 (Form 1) of PQQ.

FIG. 2 illustrates the powder XRD spectra of polymorph-2 (Form 2) of PQQ.

FIG. 3 illustrates the powder XRD spectra of polymorph-3 (Form 3) of PQQ salt.

FIG. 4 illustrates the powder XRD spectra of polymorph-4 (Form 4) of PQQ salt.

FIG. 5 illustrates the powder XRD spectra of polymorph-5 (Form 5) of PQQ salt.

FIG. 6 illustrates the powder XRD spectra of polymorph-6 (Form 6) of PQQ salt.

FIG. 7 illustrates the powder XRD spectra of polymorph-7 (Form 7) of PQQ salt.

### STATEMENT OF THE DISCLOSURE

Accordingly, the present disclosure relates to a polymorphic form of PQQ or its salt represented by formula I:

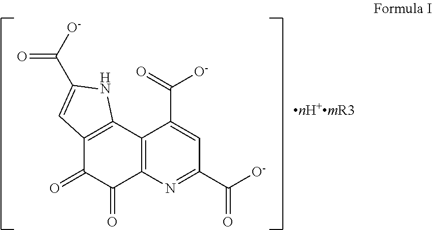

wherein “n” and “m” are selected from a group consisting of: (a) “n”=3, “m”=0 and (b) “n”=1, “m”=2, and R3 is Na⁺;
a process for the preparation of a polymorphic form of PQQ or its salt represented by formula I:

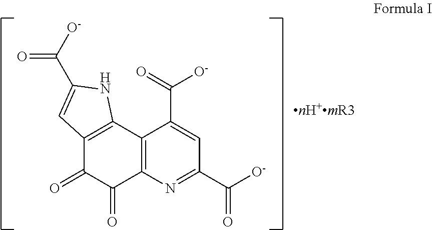

wherein “n” and “m” are selected from a group consisting of: (a) “n”=3, “m”=0 and (b) “n”=1, “m”=2, and
R3 is Na⁺, wherein said process comprises step of reacting Formula III with base followed by acid treatment to obtain the compound of Formula I

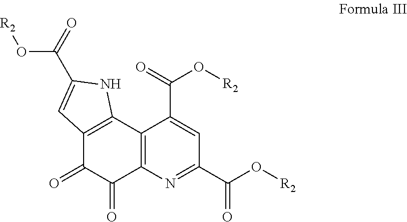

wherein, R2 is selected from a group comprising hydrogen, straight or branched chain C1-8 alkyl, straight or branched chain C1-8 alkenyl, straight or branched chain C1-8 alkynyl, aralkyl, substituted aralkyl, heteroaralkyl and substituted heteroaralkyl, and wherein each of the substituent is optionally substituted; and a composition comprising a polymorphic form of PQQ or its salt represented by formula I:

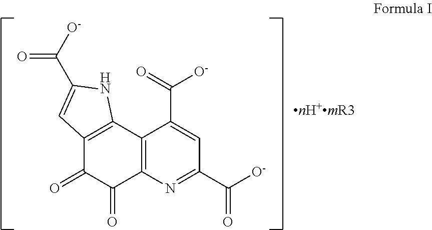

wherein “n” and “m” are selected from a group consisting of: (a) “n”=3, “m”=0 and (b) “n”=1, “m”=2, and
R3 is Na⁺,
optionally along with excipients.

### DETAILED DESCRIPTION OF THE DISCLOSURE

Accordingly, the present disclosure relates to a polymorphic form of PQQ or its salt represented by formula I:

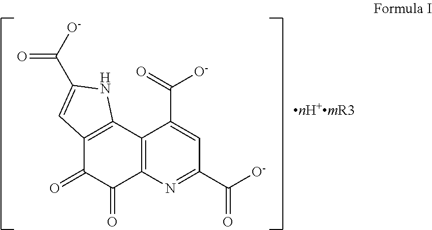

wherein “n” and “m” are selected from a group consisting of: (a) “n”=3, “m”=0 and (b) “n”=1, “m”=2; and
R3 is Na⁺.

In an embodiment of the present disclosure, when n=3 and m=0, the polymorphic form of PQQ is selected from a group comprising Form 1 with X-ray powder diffractogram pattern having characteristic peaks at diffraction angles 2θ of 7.9447±0.2°, 11.7552±0.2°, 12.6559±0.2°, 14.8219±0.2°, 16.0264±0.2°, 17.0684±0.2°, 18.8257±0.2°, 19.5474±0.2°, 22.5303±0.2°, 23.5594±0.2°, 24.7954±0.2°, 25.6632±0.2°, 27.13±0.2°, 28.3092±0.2°, 29.1776±0.2°, 30.2626±0.2°, 31.923±0.2°, 34.6208±0.2°, 35.7228±0.2°, 37.0506±0.2°, 37.8323±0.2°, 38.8985±0.2°, 39.6034±0.2°, 40.9434±0.2°, 43.9407±0.2°, 48.3058±0.2°, 54.7932±0.2°, 58.6411±0.2° and Form 2 with X-ray powder diffractogram pattern having characteristic peaks at diffraction angles 2θ of 12.5376±0.2°, 14.1135±0.2°, 15.3635±0.2°, 16.6934±0.2°, 18.0525±0.2°, 22.3898±0.2°, 25.085±0.2°, 28.2059±0.2°, 31.2156±0.2°, 35.8287±0.2°, 37.3867±0.2°, 39.5429±0.2°, 42.936±0.2°, 58.4641±0.2°.

In another embodiment of the present disclosure, the polymorphic form of salt of compound represented by Formula II is a disodium salt.

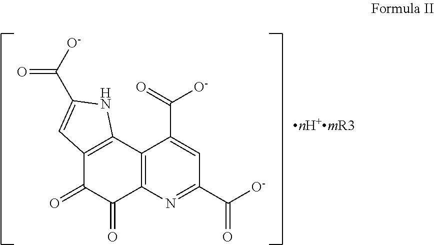

wherein “n=1” and “m=2”;
wherein, R3 is Na⁺.

In yet another embodiment of the present disclosure, when n=1, m=2, and R3 is N⁺, the polymorphic form of PQQ salt is selected from a group comprising Form 3 with X-ray powder diffractogram pattern having characteristic peaks at diffraction angles 2θ of 8.3367±0.2°, 9.5883±0.2°, 12.2471±0.2°, 15.2353±0.2°, 16.6527±0.2°, 20.989±0.2°, 22.7837±0.2°, 26.0084±0.2°, 27.4215±0.2°, 29.174±0.2°, 34.4201±0.2°, 38.7959±0.2°, Form 4 with X-ray powder diffractogram pattern having characteristic peaks at diffraction angles 2θ of 6.2526±0.2°, 8.09±0.2°, 8.5645±0.2°, 14.0915±0.2°, 17.569±0.2°, 18.6382±0.2°, 22.2638±0.2°, 23.0319±0.2°, 23.9335±0.2°, 26.4089±0.2°, 27.2276±0.2°, 28.2427±0.2°, 29.5534±0.2°, 31.7176±0.2°, 33.7511±0.2°, 34.7226±0.2°, 36.9752±0.2°, 38.8203±0.2°, 40.9029±0.2°, 43.1906±0.2°, 45.3693±0.2°, 47.3751±0.2°, Form 5 with X-ray powder diffractogram pattern having characteristic peaks at diffraction angles 2θ of 6.3087±0.2°, 8.787±0.2°, 9.4638±0.2°, 11.1383±0.2°, 12.8604±0.2°, 14.0298±0.2°, 15.1081±0.2°, 17.032±0.2°, 21.1969±0.2°, 22.3969±0.2°, 23.3678±0.2°, 26.8503±0.2°, 27.6689±0.2°, 29.435±0.2°, 31.1489±0.2°, 32.2817±0.2°, 34.0255±0.2°, 36.884±0.2°, 38.6941±0.2°, 43.2067±0.2°, 45.2776±0.2°, Form 6 with X-ray powder diffractogram pattern having characteristic peaks at diffraction angles 2θ of 8.186±0.2°, 9.4246±0.2°, 18.5305±0.2°, 26.6158±0.2°, 27.292±0.2°, 31.6378±0.2°, 45.4109±0.2°, 56.4274±0.2°, 66.1811±0.2° and Form 7 with X-ray powder diffractogram pattern having characteristic peaks at diffraction angles 2θ of 9.3426±0.2°, 11.6809±0.2°, 13.6028±0.2°, 14.9981±0.2°, 16.0269±0.2°, 18.9222±0.2°, 20.4134±0.2°, 22.0677±0.2°, 23.7113±0.2°, 25.6284±0.2°, 26.4555±0.2°, 27.4617±0.2° 28.5023±0.2°, 30.7997±0.2°, 31.6466±0.2°, 32.4609±0.2°, 35.9051±0.2°, 36.7705±0.2°, 38.0082±0.2°, 38.7336±0.2°, 41.5928±0.2°, 43.7879±0.2°, 45.3967±0.2°, 46.9161±0.2°, 48.9136±0.2°, 52.7775±0.2°, 56.5143±0.2°, 61.0903±0.2°, 66.189±0.2°.

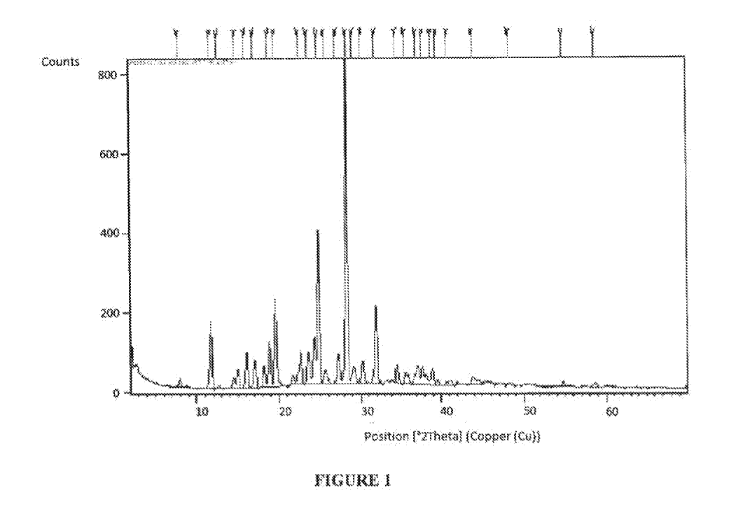

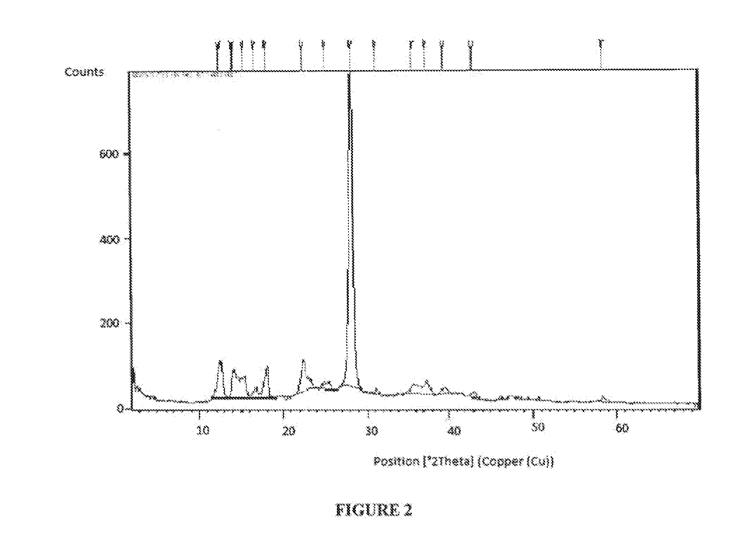

In still another embodiment of the present disclosure, wherein the Form 1 has X-ray powder diffractogram with the characteristic peaks shown in FIG. 1 and the Form 2 has X-ray powder diffractogram with the characteristic peaks shown in FIG. 2.

In still another embodiment of the present disclosure, wherein when n=1, m=2 and R3 is Na⁺, the polymorphic form of PQQ salt is selected from a group comprising Form 3, Form 4, Form 5 Form 6 and Form 7.

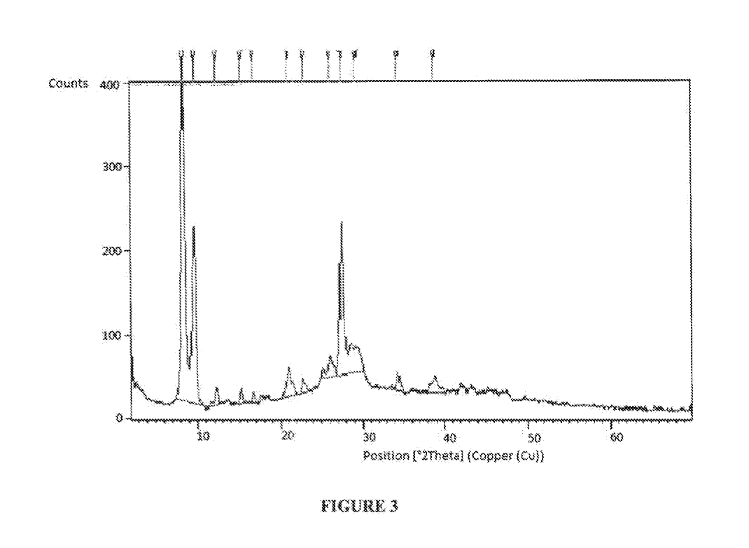

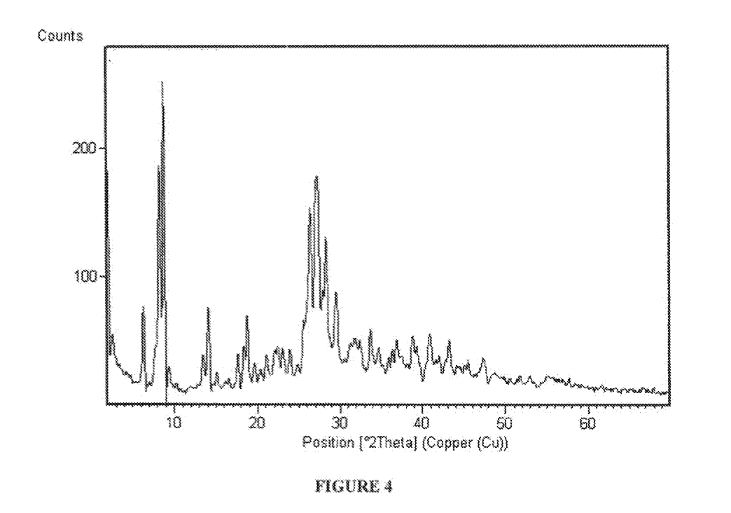

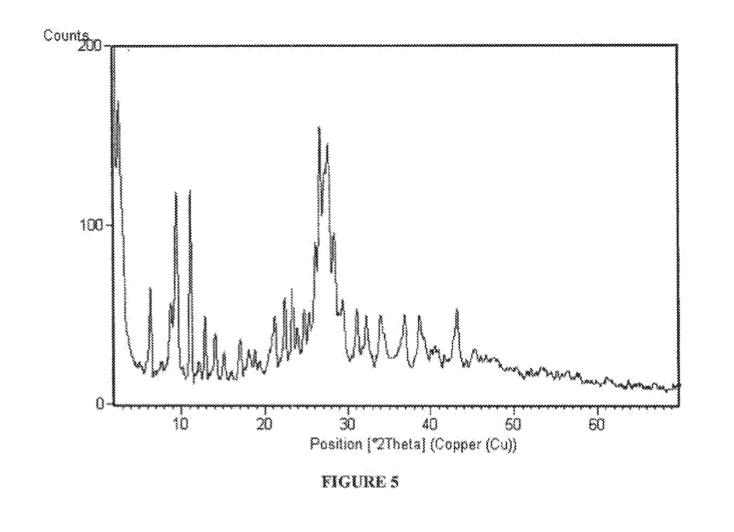

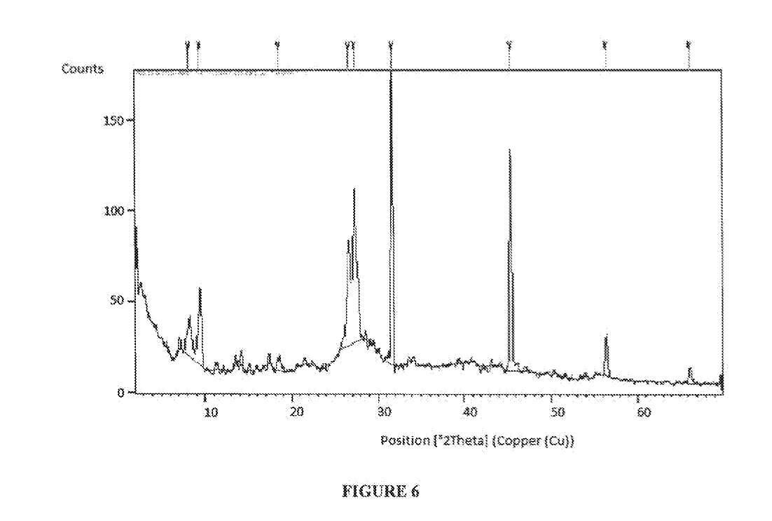

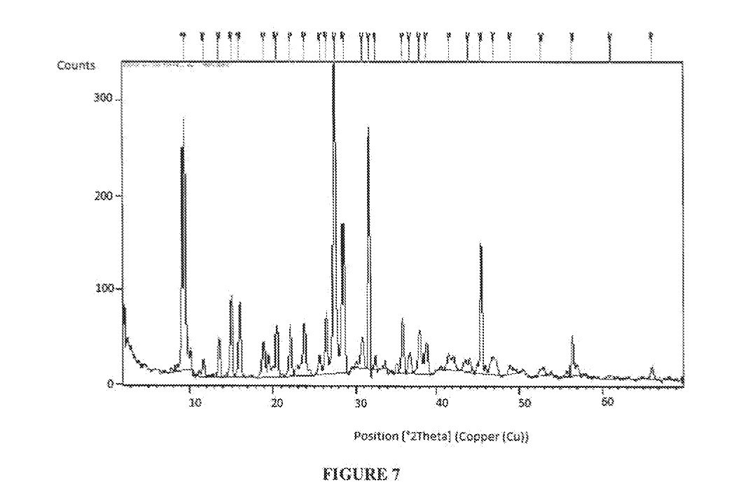

In still another embodiment of the present disclosure, wherein the Form 3 has X-ray powder diffractogram with the characteristic peaks shown in FIG. 3, the Form 4 has X-ray powder diffractogram with the characteristic peaks shown in FIG. 4, the Form 5 has X-ray powder diffractogram with the characteristic peaks shown in FIG. 5, Form 6 has X-ray powder diffractogram with the characteristic peaks shown in FIG. 6 and the Form 7 has X-ray powder diffractogram with the characteristic peaks shown in FIG. 7.

The present disclosure further relates to a process for the preparation of a polymorphic form of PQQ or its salt represented by formula I:

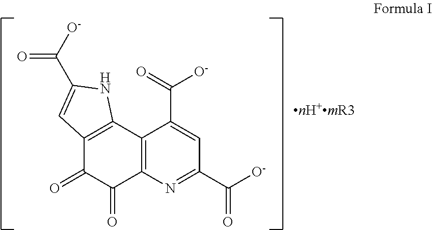

wherein “n” and “m” are selected from a group consisting of: (a) “n”=3, “m”=0 and (b) “n”=1, “m”=2; and
R3 is Na⁺, wherein said process comprises step of reacting Formula III with base followed by acid treatment to obtain the compound of Formula I

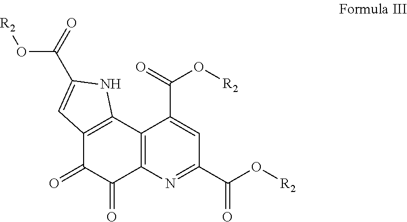

wherein, R2 is selected from a group comprising hydrogen, straight or branched chain C1-8 alkyl, straight or branched chain C1-8 alkenyl, straight or branched chain C1-8 alkynyl, aralkyl, substituted aralkyl, heteroaralkyl and substituted heteroaralkyl, and wherein each of the substituent is optionally substituted.

In an embodiment of the present disclosure, the above process is carried out in presence of base either sodium hydroxide or sodium carbonate.

In another embodiment of the present disclosure, the above process is carried out in presence of acid either hydrochloric acid or sulphuric acid.

In yet another embodiment of the present disclosure, the above process is carried out at a temperature ranging from about 10° C. to about 80° C., and for a time period ranging from about one hour to about 18 hours.

In still another embodiment of the present disclosure, the above process further comprises isolation and/or purification of the obtained pol PPQ or its salts. Further, the said isolation comprises acts selected from a group comprising, addition of solvent, quenching, filtration, and extraction and combination of acts in any order thereof.

The present disclosure further relates to a composition comprising a polymorphic form of PQQ or its salt represented by formula I:

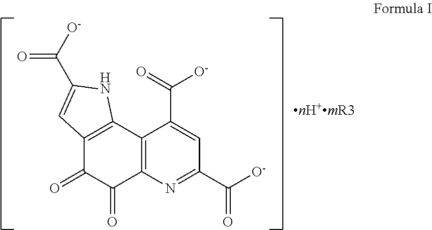

wherein “n” and “m” are selected from a group consisting of: (a) “n”=3, “m”=0 and (b) “n”=1, “m”=2; and
R3 is Na⁺,
   optionally along with excipients.

In an embodiment of the present disclosure, the composition is a nutraceutical composition or a pharmaceutical composition; and wherein the excipient is selected from a group comprising binder, disintegrant, diluent, lubricant, plasticizer, permeation enhancer and solubilizer, or any combination thereof.

In another embodiment of the present disclosure, the composition is formulated into dosage form selected from a group comprising tablet, troches, lozenges, aqueous or oily suspensions, ointment, patch, gel, lotion, dentifrice, capsule, emulsion, creams, spray, drops, dispersible powders or granules, emulsion in hard or soft gel capsules, syrups, elixirs and food supplement, or any combination thereof.

The present disclosure further relates to the use of a polymorphic form of PQQ and/or its salt represented by formula I:

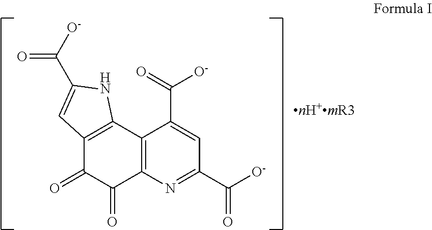

wherein “n” and “m” are selected from a group consisting of: (a) “n”=3, “m”=0 and (b) “n”=1, “m”=2; and R3 is Na⁺; for management of condition selected from a group comprising neuronal disorders, cardiovascular disorders and nutritional disorder and combinations thereof.

### Materials and Methods:

Chemicals were obtained from multiple commercial suppliers such as Apollo Scientific, Sigma-Aldrich and etc. Final purifications were carried out using Merck silica gel 230-400 mesh. TLC experiments were performed on alumina-backed silica gel 40 F254 plates (Merck, Darmstadt, Germany). The plates were illuminated under UV (254 nm) and KMnO₄. Melting points were determined using Buchi B-540 and are uncorrected. All ¹H NMR spectra were recorded on a Bruker AM-300 (300 MHz for ¹H NMR), Bruker BioSpin Corp., Germany. Molecular weights of unknown compounds were checked by LCMS 6200 series Agilent Technology. Chemical shifts are reported in ppm (δ) with reference to internal standard TMS. The signals are designated as follows: s, singlet; d, doublet; t, triplet; m, multiplet; brs, broad singlet.

The chemicals employed are as follows:
1) 4,5-dioxo-4,5-dihydro-1H-pyrrolo[2,3-f]quinoline-2,7,9-tricarboxylic acid trimethyl ester
2) Sodium hydroxide
3) Sodium carbonate
4) Hydrochloric acid
5) Water
6) Methanol
7) Ethanol

Additional embodiments and features of the present disclosure will be apparent to one of ordinary skill in art based upon description provided herein. However, the following examples should not be construed to limit the scope of the present disclosure.

### EXAMPLES

### Example 1

### Synthesis of 4,5-Dioxo-4,5-dihydro-1H-pyrrolo[2,3-f]quinoline-2,7,9-tricarboxylic acid (PQQ) polymorph—Form 1 [Formula I, ‘n’=3 and ‘m’=0]

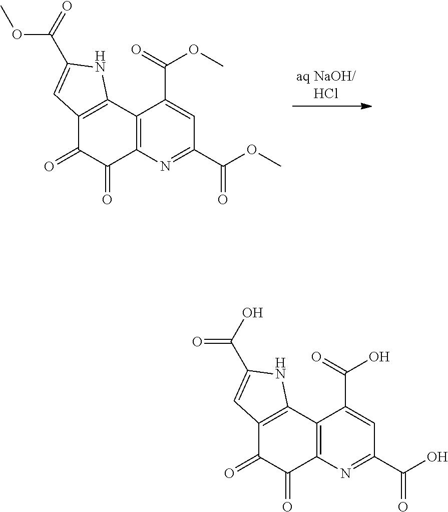

To about 15 L of about 3.5% solution of sodium hydroxide, 4,5-dioxo-4,5-dihydro-1H-pyrrolo[2,3-f]quinoline-2,7,9-tricarboxylic acid trimethyl ester (1.0 kg, 1 eq, 2.686 moles) is added at about 25-30° C. The reaction mixture is stirred at about 25° C. to about 30° C. over a period of about 3 hours. Completion of reaction is monitored by HPLC. Thereafter, the reaction mixture is acidified (pH: <1) with 12N hydrochloric acid and stirred at 40-60° C. over a period of 12 hours to precipitate the reaction mass. The precipitate is filtered to obtain product [4,5-Dioxo-4,5-dihydro-1H-pyrrolo[2,3-f]quinoline-2,7,9-tricarboxylic acid] as a bright red solid (about 0.80 Kg, Yield: 90%).

Purity by HPLC: 99.3%

¹H NMR (DMSO, 300 MHz): 7.20 (s, 1H), 8.60 (s, 1H) and 13.60 (bs, 3H)

IR (ATR, cm⁻¹) ν: 3553, 3257, 3009, 2615, 1746, 1711, 1644, 1506, 1399, 1197 and 767

### Powder XRD Spectra of Polymorph-1 of PQQ (Form 1):

The powder XRD spectra pattern of the polymorph 1 of PQQ (Form 1) is provided in FIG. 1.

### Peak List of Polymorph 1 of PQQ (Form 1)

| Pos. [°2Th.] | Height[cts] | FWHM[°2Th.] | d-spacing[Å] | Rel. Int. [%] |
|---|---|---|---|---|
| 7.9447 | 17.79 | 0.4723 | 11.12865 | 2.18 |
| 11.7552 | 166.55 | 0.3149 | 7.52840 | 20.45 |
| 12.6559 | 7.20 | 0.4723 | 6.99459 | 0.88 |
| 14.8219 | 20.43 | 0.4723 | 5.97696 | 2.51 |
| 16.0264 | 84.11 | 0.4723 | 5.53036 | 10.33 |
| 17.0684 | 69.61 | 0.3149 | 5.19501 | 8.55 |
| 18.8257 | 113.17 | 0.3149 | 4.71384 | 13.90 |
| 19.5474 | 220.15 | 0.3149 | 4.54140 | 27.03 |
| 22.5303 | 61.96 | 0.4723 | 3.94645 | 7.61 |
| 23.5594 | 81.10 | 0.3149 | 3.77634 | 9.96 |
| 24.7954 | 294.10 | 0.4723 | 3.59083 | 36.11 |
| 25.6632 | 36.75 | 0.3936 | 3.47134 | 4.51 |
| 27.1300 | 53.40 | 0.3149 | 3.28690 | 6.56 |
| 28.3092 | 814.40 | 0.3149 | 3.15261 | 100.00 |
| 29.1776 | 42.37 | 0.3936 | 3.06073 | 5.20 |
| 30.2626 | 58.40 | 0.3149 | 2.95342 | 7.17 |
| 31.9230 | 194.87 | 0.3149 | 2.80350 | 23.93 |
| 34.6208 | 45.74 | 0.3149 | 2.59096 | 5.62 |
| 35.7228 | 27.88 | 0.4723 | 2.51353 | 3.42 |
| 37.0506 | 43.58 | 0.4723 | 2.42644 | 5.35 |
| 37.8323 | 31.07 | 0.3149 | 2.37809 | 3.82 |
| 38.8985 | 42.87 | 0.3149 | 2.31532 | 5.26 |
| 39.6034 | 55.21 | 0.3149 | 2.27572 | 1.87 |
| 40.9434 | 9.12 | 0.6298 | 2.20428 | 1.12 |
| 43.9407 | 15.67 | 0.6298 | 2.06063 | 1.92 |
| 48.3058 | 5.82 | 0.4723 | 1.88414 | 0.72 |
| 54.7932 | 52.32 | 0.4723 | 1.67542 | 1.51 |
| 58.6411 | 12.31 | 0.3840 | 1.57302 | 1.51 |

### Example 2

### Synthesis of 4,5-Dioxo-4,5-dihydro-1H-pyrrolo[2,3-f]quinoline-2,7,9-tricarboxylic acid (PQQ) polymorph—Form 2 [Formula I, ‘n’=3 and ‘m’=0]

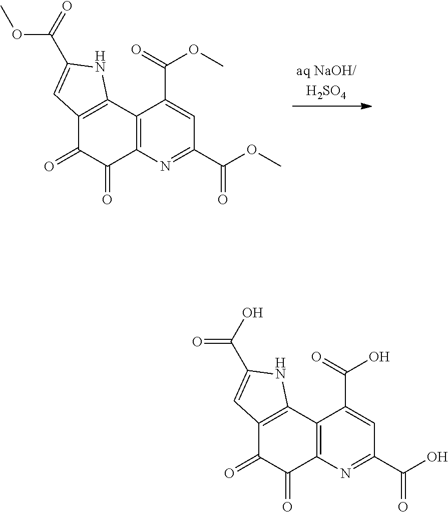

To 15 L of 3.5% solution of sodium hydroxide, 4,5-dioxo-4,5-dihydro-1H-pyrrolo[2,3-f]quinoline-2,7,9-tricarboxylic acid trimethyl ester (1.0 kg, 1 eq, 2.686 moles) is added at 25-30° C. The reaction mixture is stirred at 25° C. to 30° C. over a period of 3 hours. Completion of reaction is monitored by HPLC. Thereafter, the reaction mixture is acidified (pH: <1) with sulphuric acid and stirred at 25-30° C. over a period of 12 hours to precipitate the reaction mass. The precipitate is filtered to obtain product [4,5-Dioxo-4,5-dihydro-1H-pyrrolo[2,3-f]quinoline-2,7,9-tricarboxylic acid] as a bright red solid (about 0.78 Kg, Yield: 88%).

Purity by HPLC: 99.3%

¹H NMR (DMSO, 300 MHz): 7.02 (s, 1H), 8.60 (s, 1H) and 13.60 (bs, 3H)

IR (ATR, cm⁻¹) ν: 3503, 3255, 2965, 1746, 1711, 1644, 1506, 1320, 1196 and 766

### Powder XRD Spectra of Polymorph-2 of PQQ (Form 2):

The powder XRD spectra pattern of the polymorph 2 of PQQ (Form 2) is provided in FIG. 2.

### Peak List of Polymorph-2 of PQQ (Form 2)

| Pos. [°2Th.] | Height[cts] | FWHM[°2Th.] | d-spacing[Å] | Rel. Int. [%] |
|---|---|---|---|---|
| 12.5376 | 89.27 | 0.4723 | 7.06033 | 12.04 |
| 14.1135 | 66.97 | 0.3936 | 6.27531 | 9.03 |
| 15.3635 | 52.59 | 0.5510 | 5.76742 | 7.09 |
| 16.6934 | 25.35 | 0.4723 | 5.31085 | 3.42 |
| 18.0525 | 75.28 | 0.6298 | 4.91397 | 10.15 |
| 22.3898 | 74.80 | 0.3936 | 3.97089 | 10.08 |
| 25.0850 | 12.67 | 0.9446 | 3.55002 | 1.71 |
| 28.2059 | 741.73 | 0.3936 | 3.16393 | 100.00 |
| 31.2156 | 12.29 | 0.4723 | 2.86539 | 1.66 |
| 35.8287 | 23.53 | 0.5510 | 2.50634 | 3.17 |
| 37.3867 | 33.07 | 0.4723 | 2.40540 | 4.46 |
| 39.5429 | 13.01 | 0.7085 | 2.27906 | 1.75 |
| 42.9360 | 11.31 | 0.6298 | 2.10649 | 1.52 |
| 58.4641 | 10.21 | 0.7680 | 1.57736 | 1.38 |

### Example 3

### Synthesis of 4,5-Dioxo-4,5-dihydro-1H-pyrrolo[2,3-f]quinoline-2,7,9-tricarboxylic acid disodium (PQQ.2Na) polymorph—Form 3 [Formula II “n”=1, “m”=2]

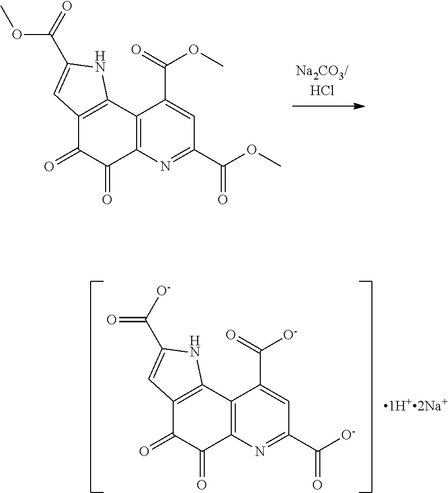

### Procedure:

To 15 L of 10% solution sodium carbonate, 4,5-dioxo-4,5-dihydro-1H-pyrrolo[2,3-f]quinoline-2,7,9-tricarboxylic acid trimethyl ester (1.0 kg, 1 eq, 2.686 moles) is added at 25-30° C. The reaction mixture is heated to 70° C. to 75° C. over a period of 16 hours. Completion of reaction is monitored by HPLC. Thereafter, the reaction mixture is acidified (pH: 3.0-3.5) with IN hydrochloric acid to precipitate the reaction mass. The precipitate is filtered at 0-5° C. to obtain the product [4,5-Dioxo-4,5-dihydro-1H-pyrrolo[2,3-f]quinoline-2,7,9-tricarboxylic acid disodium] as dark red solid (about 0.85 Kg, Yield: 85%).

¹H NMR (D₂O, 300 MHz): 6.84 (s, 1H), 8.48 (s, 1H); LC-MS (ESI): 329 (M-H), Purity by HPLC: 98.6%

IR (ATR, cm⁻¹) ν: 3407, 1719, 1666, 1621, 1580, 1537, 1497, 1356, 1243, 975 and 731

### Powder XRD Spectra of Polymorph-3 (Form 3):

The powder XRD spectra pattern of the polymorph 3 (Form 3) of the PQQ salt is provided in FIG. 3.

### Peak List of Polymorph-3 of PQQ Salt (Form 3)

| Pos. [°2Th.] | Height[cts] | FWHM[°2Th.] | d-spacing[Å] | Rel. Int. [%] |
|---|---|---|---|---|
| 8.3367 | 378.90 | 0.3149 | 10.60627 | 100.00 |
| 9.5883 | 211.31 | 0.3936 | 9.22434 | 55.77 |
| 12.2471 | 20.02 | 0.3149 | 7.22714 | 5.28 |
| 15.2353 | 13.88 | 0.4723 | 5.81569 | 3.66 |
| 16.6527 | 13.17 | 0.3936 | 5.32372 | 3.48 |
| 20.9890 | 34.09 | 0.4723 | 4.23264 | 9.00 |
| 22.7837 | 12.25 | 0.4723 | 3.90312 | 3.23 |
| 26.0084 | 24.34 | 0.4723 | 3.42604 | 6.42 |
| 27.4215 | 180.10 | 0.3936 | 3.25262 | 47.53 |
| 29.1740 | 31.57 | 0.9446 | 3.06110 | 8.33 |
| 34.4201 | 21.52 | 0.3149 | 2.60561 | 5.68 |
| 38.7959 | 18.64 | 0.7872 | 2.32121 | 4.92 |

### Example 4

### Synthesis of 4,5-Dioxo-4,5-dihydro-1H-pyrrolo[2,3-f]quinoline-2,7,9-tricarboxylic acid disodium (PQQ.2Na) polymorph—Form 4 [Formula II, “n”=1, “m”=2]

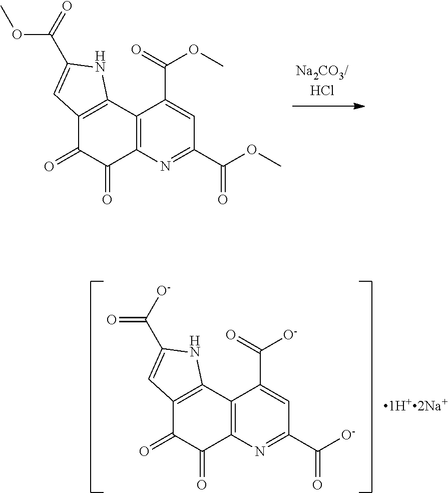

### Procedure:

To 15 L of 10% solution sodium carbonate, 4,5-dioxo-4,5-dihydro-1H-pyrrolo[2,3-f]quinoline-2,7,9-tricarboxylic acid trimethyl ester (1.0 kg, 1 eq, 2.686 moles) is added at 25-30° C. The reaction mixture is heated to 70° C. to 75° C. over a period of 16 hours. Completion of reaction is monitored by HPLC. Thereafter, the reaction mixture is acidified (pH: 3.0-3.5) with 12 hydrochloric acid to precipitate the reaction mass. The precipitate is filtered at 0-5° C. to obtain the product [4,5-Dioxo-4,5-dihydro-1H-pyrrolo[2,3-f]quinoline-2,7,9-tricarboxylic acid disodium] as dark red solid. The solid was dried at 25-30° C. over a period of 24 h under reduced pressure to attain water content is about 22%. (about 0.90 Kg, Yield: 90%).

¹H NMR (D₂O, 300 MHz): 7.07 (s, 1H), 7.69 (s, 1H); LC-MS (ESI): 329 (M-H). Purity by HPLC: 99.5%

IR (ATR, cm⁻¹) ν: 3500, 1669, 1619, 1540, 1496, 1356, 1241, and 728

### Powder XRD Spectra of Polymorph-4 of PQQ Salt (Form 4):

The powder XRD spectra pattern of the polymorph 4 of PQQ salt is provided in FIG. 4.

### Peak List of Polymorph-4 (Form 4)

### Peak List

| Pos. [°2Th.] | Height[cts] | FWHM[°2Th.] | d-spacing[Å] | Rel. Int. [%] |
|---|---|---|---|---|
| 6.2526 | 54.03 | 0.4723 | 14.13589 | 35.63 |
| 8.09 | 96.43 | 0.3149 | 10.92909 | 63.59 |
| 8.5645 | 84.08 | 0.4723 | 10.32463 | 55.45 |
| 14.0915 | 61.1 | 0.3149 | 6.28507 | 40.29 |
| 17.569 | 17.93 | 0.3149 | 5.04809 | 11.83 |
| 18.6382 | 33.79 | 0.4723 | 4.76085 | 22.28 |
| 22.2638 | 17.22 | 0.4723 | 3.99309 | 11.36 |
| 23.0319 | 17.83 | 0.3149 | 3.86163 | 11.76 |
| 23.9335 | 19.32 | 0.3149 | 3.71815 | 12.74 |
| 26.4089 | 126.44 | 0.3149 | 3.37499 | 83.38 |
| 27.2276 | 151.64 | 0.4723 | 3.27533 | 100 |
| 28.2427 | 89.84 | 0.4723 | 3.15988 | 59.25 |
| 29.5534 | 62.19 | 0.3936 | 3.02266 | 41.01 |
| 31.7176 | 26.43 | 0.9446 | 2.82118 | 17.43 |
| 33.7511 | 34.13 | 0.3149 | 2.65571 | 22.51 |
| 34.7226 | 22.18 | 0.3149 | 2.5836 | 14.63 |
| 36.9752 | 22.96 | 0.6298 | 2.43121 | 15.14 |
| 38.8203 | 33.18 | 0.3149 | 2.3198 | 21.88 |
| 40.9029 | 28.27 | 0.3149 | 2.20638 | 18.64 |
| 43.1906 | 20.23 | 0.4723 | 2.09466 | 13.34 |
| 45.3693 | 6.97 | 0.4723 | 1.99901 | 4.6 |
| 47.3751 | 17.14 | 0.576 | 1.91737 | 11.3 |

### Example 5

### Synthesis of 4,5-Dioxo-4,5-dihydro-1H-pyrrolo[2,3-f]quinoline-2,7,9-tricarboxylic acid disodium (PQQ.2Na) polymorph—Form 5 [Formula II, “n”=1 “m”=2]

The product obtained in example 4, was further dried at 25-30° C. to attain moisture content about 12%.

The below spectral data corresponds to PQQ with 12% moisture content.

### Peak List

| Pos. [°2Th.] | Height[cts] | FWHM[°2Th.] | d-spacing[Å] | Rel. Int. [%] |
|---|---|---|---|---|
| 6.3087 | 42.06 | 0.3149 | 14.01033 | 38.76 |
| 8.787 | 36.31 | 0.3149 | 10.06366 | 33.46 |
| 9.4638 | 99.09 | 0.3149 | 9.34542 | 91.31 |
| 11.1383 | 94.35 | 0.3149 | 7.94394 | 86.94 |
| 12.8604 | 30.31 | 0.3149 | 6.88381 | 27.93 |
| 14.0298 | 22.3 | 0.4723 | 6.31257 | 20.55 |
| 15.1081 | 14.35 | 0.3149 | 5.86436 | 13.23 |
| 17.032 | 19.52 | 0.3149 | 5.20602 | 17.99 |
| 21.1969 | 26.59 | 0.3149 | 4.19159 | 24.5 |
| 22.3969 | 28.59 | 0.4723 | 3.96965 | 26.35 |
| 23.3678 | 31.12 | 0.4723 | 3.80687 | 28.68 |
| 26.8503 | 92.63 | 0.4723 | 3.3205 | 85.36 |
| 27.6689 | 108.52 | 0.3149 | 3.22409 | 100 |
| 29.435 | 29.71 | 0.3149 | 3.03455 | 27.38 |
| 31.1489 | 28.06 | 0.3149 | 2.87138 | 25.86 |
| 32.2817 | 24.94 | 0.4723 | 2.77316 | 22.99 |
| 34.0255 | 24.44 | 0.3149 | 2.63492 | 22.53 |
| 36.884 | 25.49 | 0.3149 | 2.43702 | 23.49 |
| 38.6941 | 25.47 | 0.3149 | 2.32708 | 23.47 |
| 43.2067 | 29.81 | 0.3149 | 2.09392 | 27.47 |
| 45.2776 | 7.82 | 0.864 | 2.00119 | 7.2 |

Note:
When water content is 12%, there is a new peak appeared at 2θ of 11.139.

¹H NMR (D₂O, 300 MHz): 6.97 (s, 1H), 8.48 (s, 1H); LC-MS (ESI): 329 (M-H), Purity by HPLC: 99.5%

IR (ATR, cm⁻¹) ν: 3500, 3408, 1669, 1621, 1497, 1359, 1241 and 729

### Example 6

### 4,5-Dioxo-4,5-dihydro-1H-pyrrolo[2,3-f]quinoline-2,7,9-tricarboxylic acid disodium (PQQ.2Na) polymorph—Form 6 [Formula II, “n”=1, “m”=2]

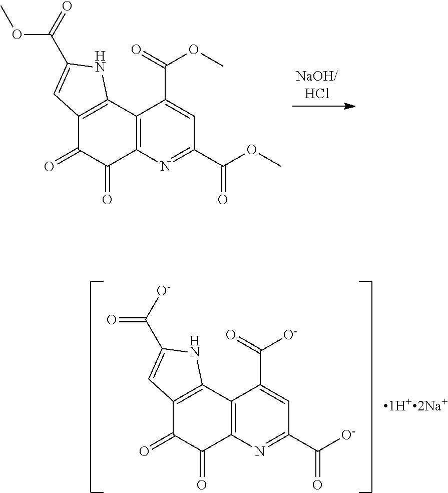

### Procedure:

To 15 L of 3.5% solution of sodium hydroxide, 4,5-dioxo-4,5-dihydro-1H-pyrrolo[2,3-f]quinoline-2,7,9-tricarboxylic acid trimethyl ester (1.0 kg, 1 eq, 2.68 mol) is added at 25° C.-30° C. and stirred over a period of 16 h. Completion of reaction is monitored by HPLC. Thereafter, the reaction mixture is acidified (pH: 3.0-3.5) with 12N hydrochloric acid over a period of 1 h to precipitate the precipitate the reaction mass. The precipitate is filtered at 20° C.-30° C. to obtain product [4,5-Dioxo-4,5-dihydro-1H-pyrrolo[2,3-f]quinoline-2,7,9-tricarboxylic acid disodium] as dark red solid (about 0.84 Kg, 84%).

¹H NMR (D₂O, 300 MHz): 6.84 (s, 1H), 8.48 (s, 1H); LC-MS (ESI): 329 (M-H), Purity by HPLC: 99.4%

IR (ATR, cm⁻¹) ν: 3423, 2558, 1717, 1674, 1611, 1543, 1502, 1235, 1147, 938 and 718

### Powder XRD Spectra of Polymorph-5 of PQQ Salt (Form 6):

The powder XRD spectra pattern of the polymorph 6 is provided in FIG. 6.

| Pos. [°2Th.] | Height[cts] | FWHM[°2Th.] | d-spacing[Å] | Rel. Int. [%] |
|---|---|---|---|---|
| 8.1860 | 19.08 | 0.4723 | 10.80107 | 13.21 |
| 9.4246 | 42.36 | 0.3149 | 9.38425 | 29.33 |
| 18.5305 | 7.84 | 0.4723 | 4.78826 | 5.43 |
| 26.6158 | 56.41 | 0.3149 | 3.34922 | 39.06 |
| 27.2920 | 84.53 | 0.3149 | 3.26776 | 58.53 |
| 31.6378 | 144.43 | 0.4723 | 2.82812 | 100.00 |
| 45.4109 | 112.58 | 0.4723 | 1.99728 | 77.95 |
| 56.4274 | 23.86 | 0.3149 | 1.63071 | 16.52 |
| 66.1811 | 8.93 | 0.3840 | 1.41090 | 6.18 |

### Peak List of Polymorph-6:

### Example 7

### 4,5-Dioxo-4,5-dihydro-1H-pyrrolo[2,3-f]quinoline-2,7,9-tricarboxylic acid disodium (PQQ.2Na) polymorph—Form 7 [Formula II, “n”=1, “m”=2]

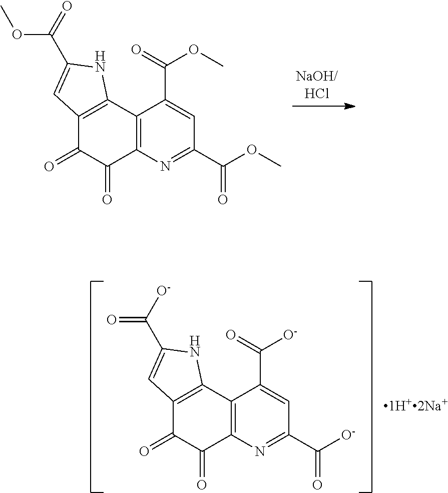

### Procedure:

To 15 L of 3.5% solution of sodium hydroxide, 4,5-dioxo-4,5-dihydro-1H-pyrrolo[2,3-f]quinoline-2,7,9-tricarboxylic acid trimethyl ester (1.0 kg, 1 eq, 2.686 moles) is added at 25-30° C. The reaction mixture was heated to 25 to 30° C. over a period of 16 h Completion of reaction is monitored by HPLC. Thereafter, the reaction mixture is acidified (pH: 3.0-3.5) with 12N hydrochloric acid over a period of 3 h under vigorous stirring to precipitate the reaction mass. The precipitate is filtered at 0-5° C. to obtain product [4,5-Dioxo-4,5-dihydro-1H-pyrrolo[2,3-f]quinoline-2,7,9-tricarboxylic acid disodium] as dark red solid (about 0.88 Kg, 88%).

¹H NMR (D₂O, 300 MHz): 6.84 (s, 1H), 8.48 (s, 1H); LC-MS (ESI): 329 (M-H), Purity by HPLC: 99.4%

IR (ATR, cm-1) ν: 3414, 2474, 1681, 1643, 1503, 1356, 1296, 1238, 1084, 1049, 811, 723 and 698

### Powder XRD Spectra of Polymorph-6 of PQQ Salt (Form 7):

The powder XRD spectra pattern of the polymorph 7 is provided in FIG. 7.

### Peak List of Polymorph-7

| Pos. [° 2Th.] | Height[cts] | FWHM[°2Th.] | d-spacing[Å] | Rel. Int. [%] |
|---|---|---|---|---|
| 9.3426 | 251.02 | 0.4723 | 9.46644 | 76.02 |
| 11.6809 | 15.80 | 0.4723 | 7.57611 | 4.78 |
| 13.6028 | 41.11 | 0.3149 | 6.50976 | 12.45 |
| 14.9981 | 77.18 | 0.3149 | 5.90713 | 23.37 |
| 16.0269 | 72.44 | 0.4723 | 5.53017 | 21.94 |
| 18.9222 | 38.41 | 0.3149 | 4.69003 | 11.63 |
| 20.4134 | 46.95 | 0.4723 | 4.35067 | 14.22 |
| 22.0677 | 52.94 | 0.3149 | 4.02812 | 16.03 |
| 23.7113 | 50.53 | 0.4723 | 3.75250 | 15.30 |
| 25.6284 | 20.91 | 0.3149 | 3.47597 | 6.33 |
| 26.4555 | 63.79 | 0.3149 | 3.36915 | 19.32 |
| 27.4617 | 330.21 | 0.3149 | 3.24795 | 100.00 |
| 28.5023 | 157.52 | 0.3149 | 3.13169 | 47.70 |
| 30.7997 | 32.81 | 0.3149 | 2.90313 | 9.94 |
| 31.6466 | 228.13 | 0.3149 | 2.82735 | 69.09 |
| 32.4609 | 14.74 | 0.3936 | 2.75826 | 4.46 |
| 35.9051 | 57.91 | 0.3149 | 2.50118 | 17.54 |
| 36.7705 | 20.77 | 0.3936 | 2.44428 | 6.29 |
| 38.0082 | 46.45 | 0.3149 | 2.36748 | 14.07 |
| 38.7336 | 33.15 | 0.3936 | 2.32480 | 10.04 |
| 41.5928 | 15.95 | 0.6298 | 2.17136 | 4.83 |
| 43.7879 | 9.27 | 0.7872 | 2.06747 | 2.81 |
| 45.3967 | 126.11 | 0.4723 | 1.99787 | 38.19 |
| 46.9161 | 18.71 | 0.7872 | 1.93665 | 5.67 |
| 48.9136 | 9.44 | 0.4723 | 1.86214 | 2.86 |
| 52.7775 | 7.94 | 0.4723 | 1.73455 | 2.41 |
| 56.5143 | 35.19 | 0.4723 | 1.62841 | 10.66 |
| 61.0903 | 3.33 | 0.9446 | 1.51694 | 1.01 |
| 66.1890 | 13.72 | 0.3840 | 1.41075 | 4.15 |

1-10. (canceled)

11. A polymorphic form of PQQ or its salt represented by formula I: 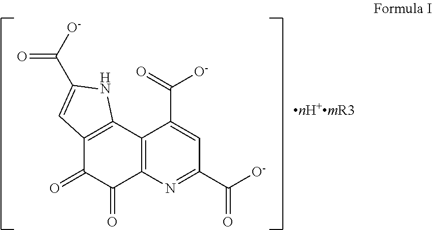
wherein “n” and “m” are selected from a group consisting of: (a) “n”=3, “m”=0 and (b) “n”=1, “m”=2; and
R3 is Na⁺.

12. The polymorphic form as claimed in claim 11, wherein when n=1, m=2, and R3 is Na⁺, the polymorphic form of PQQ salt is selected from a group comprising Form 3 with X-ray powder diffractogram pattern having characteristic peaks at diffraction angles 2θ of 8.3367±0.2°, 9.5883±0.2°, 12.2471±0.2°, 15.2353±0.2°, 16.6527±0.2°, 20.989±0.2°, 22.7837±0.2°, 26.0084±0.2°, 27.4215±0.2°, 29.174±0.2°, 34.4201±0.2°, 38.7959±0.2°, Form 4 with X-ray powder diffractogram pattern having characteristic peaks at diffraction angles 2θ of 6.2526±0.2°, 8.09±0.2°, 8.5645±0.2°, 14.0915±0.2°, 17.569±0.2°, 18.6382±0.2°, 22.2638±0.2°, 23.0319±0.2°, 23.9335±0.2°, 26.4089±0.2°, 27.2276±0.2°, 28.2427±0.2°, 29.5534±0.2°, 31.7176±0.2°, 33.7511±0.2°, 34.7226±0.2°, 36.9752±0.2°, 38.8203±0.2°, 40.9029±0.2°, 43.1906±0.2°, 45.3693±0.2°, 47.3751±0.2°, Form 5 with X-ray powder diffractogram pattern having characteristic peaks at diffraction angles 2θ of 6.3087±0.2°, 8.787±0.2°, 9.4638±0.2°, 11.1383±0.2°, 12.8604±0.2°, 14.0298±0.2°, 15.1081±0.2°, 17.032±0.2°, 21.1969±0.2°, 22.3969±0.2°, 23.3678±0.2°, 26.8503±0.2°, 27.6689±0.2°, 29.435±0.2°, 31.1489±0.2°, 32.2817±0.2°, 34.0255±0.2°, 36.884±0.2°, 38.6941±0.2°, 43.2067±0.2°, 45.2776±0.2°, Form 6 with X-ray powder diffractogram pattern having characteristic peaks at diffraction angles 2θ of 8.186±0.2°, 9.4246±0.2°, 18.5305±0.2°, 26.6158±0.2°, 27.292±0.2, 31.6378±0.2°, 45.4109±0.2°, 56.4274±0.2°, 66.1811±0.2°.

13. A process for the preparation of a polymorphic form of PQQ or its salt represented by formula I: 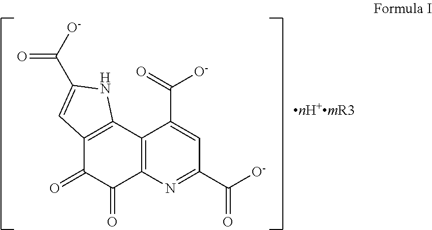
wherein “n” and “m” are selected from a group consisting of: (a) “n”=3, “m”=0 and (b) “n”=1, “m”=2; and
R3 is Na⁺,
wherein said process comprises step of reacting Formula III with base followed by acid treatment to obtain the compound of Formula I 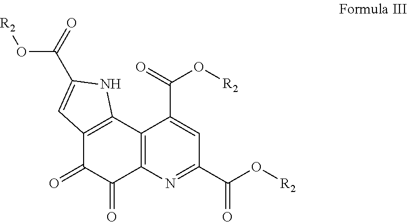
wherein, R2 is selected from a group comprising hydrogen, straight or branched chain C1-8 alkyl, straight or branched chain C1-8 alkenyl, straight or branched chain C1-8 alkynyl, aralkyl, substituted aralkyl, heteroaralkyl and substituted heteroaralkyl, and wherein each of the substituent is optionally substituted.

14. The process as claimed in claim 13, wherein the base is sodium hydroxide or sodium carbonate.

15. The process as claimed in claim 13, wherein the acid is hydrochloric acid or sulphuric acid.

16. The process as claimed in claim 13, wherein said process is carried out at a temperature ranging from about 10° C. to about 80° C., and for a time period ranging from about one hour to about 18 hours.

17. A composition comprising a polymorphic form of PQQ or its salt represented by formula I: 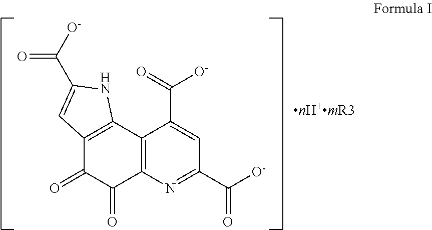
wherein “n” and “m” are selected from a group consisting of: (a) “n”=3, “m”=0 and (b) “n”=1, “m”=2; and
R3 is Na⁺,
optionally along with excipients.

18. The composition as claimed in claim 17, wherein the composition is a nutraceutical composition or a pharmaceutical composition; and wherein the excipient is selected from a group comprising binder, disintegrant, diluent, lubricant, plasticizer, permeation enhancer and solubilizer, or any combination thereof.

19. The composition as claimed in claim 17, wherein the composition is formulated into dosage form selected from a group comprising tablet, troches, lozenges, aqueous or oily suspensions, ointment, patch, gel, lotion, dentifrice, capsule, emulsion, creams, spray, drops, dispersible powders or granules, emulsion in hard or soft gel capsules, syrups, elixirs and food supplement, or any combination thereof.
